(19) United States
(12) Patent Application Publication
(10) Pub. No.: US 2024/0274284 A1
(43) Pub. Date: Aug. 15, 2024

(54) SYSTEM AND METHOD FOR COLLECTING AND OPTIMIZING MEDICAL DATA

(71) Applicant: CAT AI LTD, Herzliya (IL)

(72) Inventors: Avner ROUACH, Udim (IL); Shlomo-Chanan ISRAELIT, Haifa (IL); Avi MOTOVA, Haifa (IL)

(21) Appl. No.: 18/619,906

(22) Filed: Mar. 28, 2024

Related U.S. Application Data
(63) Continuation-in-part of application No. PCT/IL2022/051033, filed on Sep. 29, 2022.
(60) Provisional application No. 63/249,936, filed on Sep. 29, 2021.

(51) Int. Cl. G16H 40/67; A61B 5/00; G06F 9/451; G06T 7/00; G06V 20/62; G06V 30/10; G16H 10/60; G16H 50/30

(52) U.S. Cl. CPC G16H 40/67; A61B 5/444; A61B 5/7275; A61B 5/7435; G06F 9/453; G06T 7/0012; G06V 20/62; G06V 30/10; G16H 10/60; G16H 50/30; G06T 2200/24; G06T 2207/10024; G06T 2207/30096; G06T 2207/30168

(57) ABSTRACT

A system and method for acquiring medical data from connected and non-connected devices, and optimizing said data, comprising:
a mobile device containing at least one processor, a memory unit and at least one camera, a server containing at least one processor and a memory unit, wherein:
said mobile device takes an image of a non-connected health monitoring device, transferring the data to a server, said server using computational means to detect the type and data from said device using machine learning modules, categorizing said results and saving the correct results into a database.

### CROSS REFERENCE TO RELATED APPLICATIONS

This application is a Continuation-in-Part of International Application No. PCT/IL2022/051033 having International filing date of Sep. 29, 2022, which claims the benefit of priority of U.S. Provisional Patent Application No. 63/249,936, filed Sep. 29, 2021, the contents of which are all incorporated herein by reference in their entirety.

### FIELD OF INVENTION

The present disclosure relates to systems collecting personal medical data, more specifically, it relates to collecting medical data from both connected and unconnected devices and their optimization to provide accurate user medical information. Said information can be used to give personal predictions and suggestions regarding health.

### BACKGROUND OF INVENTION

More and more people wear web-connected devices that have health data monitors built into them. Most common-smart watches with heart-rate monitors. According to one article, the usage of such monitoring devices has more than tripled in the past four years. The latest versions of smart watches also have blood-oxygen saturation sensors, sleep tracking, activity tracking, etc. These devices will proliferate and will certainly include new sensors at each new iteration to better monitor health and activity.

The problem with these devices is that all currently used devices are calibrated with data from healthy users. It is known that each of us has different “normal” measurements when it comes to heart rate or other various health related datapoints. In addition, specific data that cannot yet be measured by connected devices can affect the data returns from said connected devices (i.e. a user with high blood pressure might have different risks at high heart rate levels than a user with normal blood pressure values).

U.S. Pat. No. 8,180,652 describes a remote health care system for gathering data from devices, storing the data in a database, then using historical data as a comparison point, giving a list of risk-factors of diseases based on symptom lists. This patent does deal with the gathering of data from devices but does not differentiate between connected devices such as smartwatches and bracelets, to data from non-connected home health measuring devices, such as blood pressure monitors, nor does it explain if it can get data from such devices in any means other than manually typing the data.

U.S. Pat. No. 10,614,569 describes a system that takes a picture of a non-connected health measurement device, grabs the data off it, transcribes it and saves the relevant data in a database. However, this patent uses a repository of specific health measurement monitors, where they have a grid layout for each device. Using the grid laid out over the image of the monitoring device allows them to take specific data and translate it to machine language. This system supports only a few monitoring devices saved and analyzed in a repository and cannot handle multiple devices, made by a plurality of manufacturers to different standards, with new versions coming out to market every year.

In view of the above, there is still an unmet long-felt need for a system that will be able to collect health data from a specific user, from both connected and non-connected devices, and optimize that information for better health valuation, risk-factor analysis, etc.

### SUMMARY OF THE INVENTION

It is thus one object of the present invention to disclose a system useful for acquiring medical data from connected and non-connected devices, and optimizing said data, comprising a mobile device containing at least one processor, a memory unit and at least one camera, a server containing at least one processor and a memory unit.

Said system will receive an image of a non-connected health monitoring device from said mobile device, transfer the data to a server, said server using computational means to detect the type and data from said device using machine learning modules, categorizing said results and saving the correct results into a database.

Said system can be configured to receive images from a specific application on a mobile device, connected to a specific user, or from a messaging service, connected to a specific user.

The system can also be configured to send reminders, suggestions and predictions using an automatic chatbot or other to a messaging service connected to a specific user.

The system can be further configured to receive prior health conditions from a user, adjusting gathered data, as well as adjusting given suggestions and predictions given by the system to the user.

The system can be further configured to detect parameters for a user, apply it against certain protocols to check for specific health issues, like patient deterioration (using the NEWS protocol), the system can then report the condition to the user, as well as others (e.g. family members).

It is another object of the present invention to disclose a method for acquiring medical data from connected and non-connected devices, and optimizing said data, where the system receives an image from a user, the system concurrently runs identification of the image against a machine learning module to detect a specific type of a non-connected health monitoring device and detecting and extracting text from the image. the system further decides on the correct monitoring device and text and saves said data in a database.

The method can be configured to receive images of non-connected devices from a specific application on a mobile device, connected to a specific user, or from a messaging service, connected to a specific user.

The method can also be configured to send reminders, suggestions and predictions using an automatic chatbot or other to a messaging service connected to a specific user.

The method can be further configured to receive prior health conditions from a user, adjusting gathered data, as well as adjusting given suggestions and predictions given by the method to the user.

The method can be further configured to detect parameters for a user, apply it against certain protocols to check for specific health issues, like patient deterioration (using the NEWS protocol), the system can then report the condition to the user, as well as others (e.g. family members).

It is another object of the present invention to describe a method for optimizing a user collected health data where data is collected about a user from both connected and non-connected devices, optimal data is continuously computed, correcting data received from connected devices to get accurate health data for a specific user.

It is another object of the present invention to describe a method to further optimize data received from both connected and non-connected devices to achieve an accurate health status reading for a specific user.

It is another object of the present invention to describe a method for normalizing data based on prior data collected about a user from both connected wearable devices and non-connected devices.

It is another object of the present invention to use said accurate data to provide alerts, predictions and suggestions regarding the health status and issues of a specific individual.

It is another object of the invention to provide a method for analyzing a medical condition performed by a computer. The method comprises steps of providing, by a computer, a graphical user interface (GUI) wizard on a mobile phone, the wizard comprising instructions for taking pictures of different areas of the body using an AI model to recognize body areas, imaging vital sign devices, and checking lighting and sharpness of said images, real time identification of lesions by size and color, qualifying images and sending the qualified images to the algorithm server for evaluating degree of risk that the lesion is cancerous.

### BRIEF DESCRIPTION OF THE FIGURES

The accompanying drawings, which are included to provide a further understanding of the invention and are incorporated in and constitute a part of this specification, illustrate embodiments of the invention and together with the description serve to explain the principles of the invention.

FIG. 1 depicting a schematic presentation of the data retrieval system;

FIG. 2 depicting a flow chart of the method of data parsing from an image;

FIG. 3 depicting a flow chart of the training model for the health monitoring device identification module;

FIG. 4 depicting a flow chart of the system for optimizing data from connected and non-connected devices;

FIG. 5 shows details of a validation of system and methods of the present invention.

FIG. 6 shows details of a method for analyzing a medical condition performed by a computer.

### DETAILED DESCRIPTION OF PREFERRED EMBODIMENTS

As used herein, the term “NEWS protocol” refers to the national early warning score—a protocol by the royal college of physicians regarding standardization of assessment and response to acute illness.

The following description is provided, alongside all chapters of the present invention, so as to enable any person skilled in the art to make use of the invention and sets forth the best modes contemplated by the inventor of carrying out this invention. Various modifications, however, are adapted to remain apparent to those skilled in the art, since the generic principles of the present invention have been defined specifically to provide embodiments of a system that acquires medical data from both connected wearable devices, process said information to continuously gain more accurate information, and use said information to send suggestions, predictions and reminders to a user, either via specific application or through a messaging service.

The system can be connected to many devices and can learn to read information from many non-connected health monitors, both presently existing as well as new models and new monitors. The present invention's system and method is configured to be used by individuals in a home environment, though it can also be used by medical professionals.

The term connected devices refers hereinafter to wearable devices that monitor one or more vital or health related data from a user and can connect to a network and save that information independently.

The term non-connected devices refer hereinafter to medical devices that monitor one or more vital or health related data from a user and are not connected to a network.

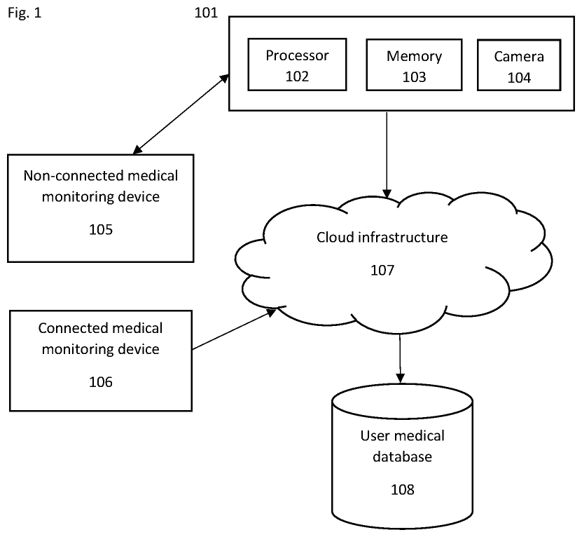

FIG. 1 shows a schematic representation of the system, wherein a mobile device (101), containing at least one processor (102), one memory (103) and a camera (104). The mobile device takes a picture of a non-connected medical monitoring device (105), that sends the data to the cloud (107) for data extraction, which is then saved in a database (108). Connected medical monitoring devices (106) are connected to the cloud and the data from there is automatically saved in the database.

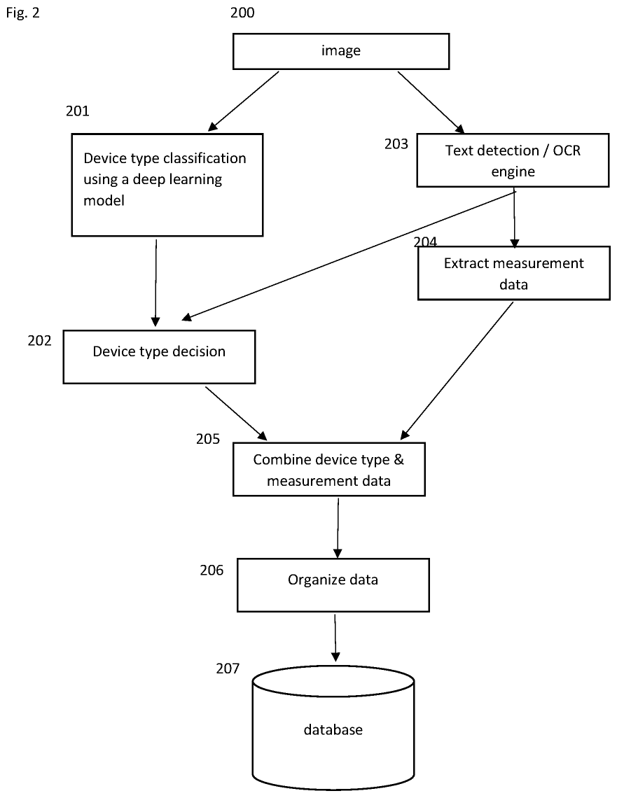

FIG. 2 shows a flow chart of the system's image parsing operation. The system receives an image from (200), which causes two concurrent threads to start. One handles the device type classification (201). The second uses OCR and text detection (203) to get text data from the image, the text can contain both regular text and seven-segment text, the text is then extracted (204). The OCR text detection (203) method also sends data, together with the device type classification model (201) to get a device type decision (202). The system combines both the device type and the text extracted to decide the exact data type and measurements received from said devices (205). The system then organizes the data for saving into a database (206) and saves it on the database (207).

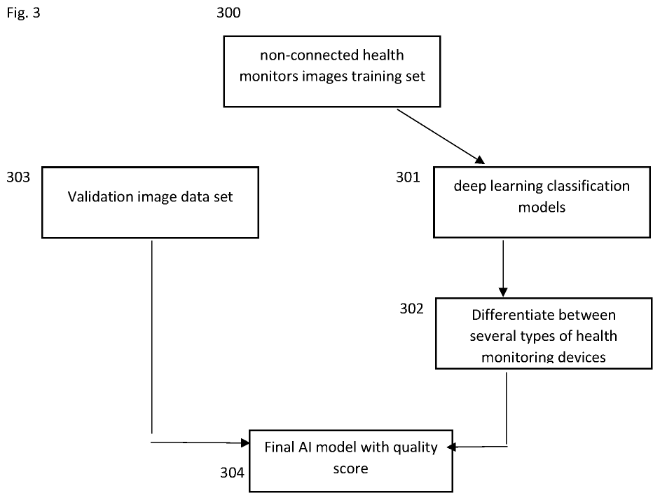

FIG. 3 shows a flow chart of the basic machine learning training for the detection of types of non-connected health monitors. The system starts with an image data set of different non-connected health monitors (300). The system feeds these images to a deep learning module (e.g. efficientnet-B4) (301). The system learns to differentiate between several types of non-connected health monitors: e.g. blood pressure, oxygen saturation, glucose, temperature, weight, UK (302). The system then uses a validation set of images (303) against the deep learning model, to finally create an AI model and quality score (304) for identifying specific types of non-connected health monitors according to specific types.

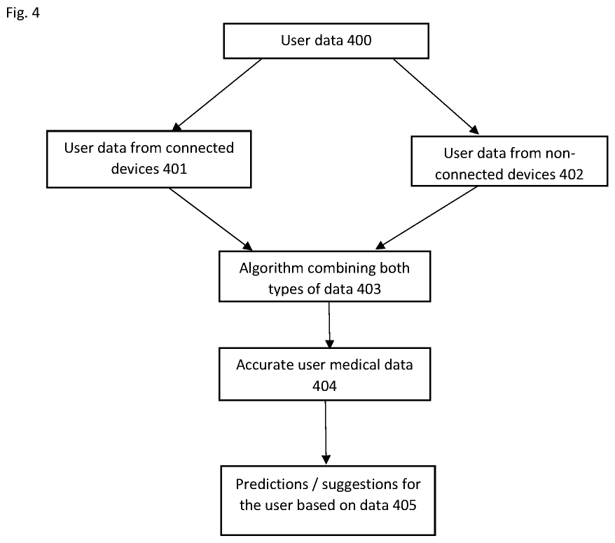

FIG. 4 shows a flow chart of the optimization method the system uses to get correct user data from both connected and non-connected devices. The system starts with data from the database, which is received from both connected (401) and non-connected devices (402). Connected devices are off the shelf items that contain sensors that have been tested only on healthy users, the data taken from connected devices flows regularly, but is inaccurate. The system uses an algorithm (403) to combine both the data from connected devices to the data received through the system from non-connected devices. The accurate data (404) can then be used for future predictions and health suggestions (405). Reference will now be made in detail to the preferred embodiments of the present invention, examples of which are illustrated in the accompanying drawings.

Reference is now made to an embodiment of the present invention, disclosing a method useful for detecting and extracting medical data from non-connected health monitoring devices. The system is first trained to detect a type of a non-connected health monitoring device out of a few specific options. The system then receives and image and concurrently checks the image for the type of health monitoring device, and an OCR and text detection process to detect and extract the text that is written on the face of the measurement device. The system then combines both text data and the image device classification to accurately determine what the measurement device is and what the data/measurement data types are. For example, a blood pressure measurement device will have specific data types and measurement limits: systolic (120 in healthy adults), diastolic (80 in healthy adults), heart rate (60-90 at rest). After correctly deciding on the correct data—it is parsed and saved onto a database.

Reference is now made to an embodiment of the present invention disclosing a method useful for receiving images from users for analysis. A user can send the image in several ways. In any case, a user first must register into the system, and can then download a specific application that is connected to the service. In this case the application will connect to the mobile device camera to take a picture and send it to the system for analysis and data extraction. Additionally, a user can send an image to the system using a messaging service (e.g. whatsapp, snapchat, etc.), the system can have a dedicated account in one or more messaging services, and receive images from there-connecting each image to a specific user based on the user's phone number or other identity information provided at the registration phase.

Reference is now made to an embodiment of the present invention disclosing a method useful for optimizing health data. The system can be connected to a multitude of connected wearable connected health monitoring devices (e.g. smart watches, bracelets, etc.). These devices are calibrated towards healthy individuals, but continuously sends out information about a user to be stored in a database. This data is not always accurate. To get more accurate information, the system also receives updates from non-connected health monitoring devices, optimizing both types of data to get accurate health reports regarding a specific user. Said data can then be used for alerts, predictions, and health suggestions.

Reference is now made to an embodiment of the system where a user can enter specific prior health conditions, allowing the system to further optimize health data regarding potential risks, health readings, predictions, and suggestions.

Reference is now made to an embodiment where the system uses a chatbot or other, connected to the user's messaging service (e.g. whatsapp, iMessage, snapchat, etc.), to send ongoing health suggestions to the user.

Reference is made to an embodiment where the system can automatically collect data and calculate the results against the NEWS protocol for determining patient deterioration. The system can then send alerts to the user, as well as others (e.g. family members, caregivers), regarding the deterioration of his condition, including suggestions and reminders to see a physician, etc.

### Examples

Example given to a method of normalizing data received from wearable connected device based on data collected about a user from both connected and non-connected device. For example, a smartwatch may continuously check heart rate measurements from an individual user, but said measurements are mostly inaccurate. However, blood pressure monitors also automatically check heartrate in a more precise fashion. When comparing rest heartrate from both the connected smartwatch and the non-connected blood pressure monitor, the system can learn to recognize the differential values and automatically normalize the data coming from the connected smartwatch, to give correct values.

In another example, the system follows information received by a user's connected and non-connected devices and sends out messages via a messaging service. For example, the system can send hourly reminders to get up, stretch, drink water. The system can also remind a user to take medicine, exercise, etc. The same system can be configured for many reminder options, to make sure the user gets the reminders he wants to see.

In another example the system can use data collected and apply it against the NEWS protocol-a protocol used to check the deterioration of a patient suffering from a particular illness. A person with heart failure, who regularly sends body weight information to the system, can be alerted regarding weight increases (reflecting fluid retention) before a severe clinical state (pulmonary edema) appears. As another example, Covid patients who continuously sends oxygen saturation data to the system can be alerted if the oxygen saturation is decreasing over time, which can lead to dangerous respiratory insufficiency. Now these follow ups are done only by nurses, and so are only conducted when a patient is in full care in a hospital or a similar facility. This system will allow a user to get the same check results at home.

In another example, an embodiment of the present invention comprises a software wizard or setup assistant. This is a user that leads the user through a sequence of small steps, to configure a program for the first time.

The following describes the wizard analysis procedure:
Mobile phone:
Based on the image and the user's meta data, a distinction is made for the type of processing required. Once this has been done, a dedicated wizard is run, according to the classification obtained. The wizard provides instructions and assistance to the user for continuing the process.

### Example

1. Oral cancer, Instructions are given for taking pictures in different areas of the mouth using an AI model to recognize faces and facial parts.
2. Vital sign devices, we run real time OCR to check the sharpness of the image and the lighting.
3. Wound Analysis, Identifying the size and color calibration sticker automatically in real time as a condition for sending the image.
Reference is now made to the

Backend Server:

According to the data sent by the user and classify by the wizard AI models are run to perform a complete analysis for data extraction.

### Exemplary Steps:

1. Oral cancer-AI Model Classification on each image and analysis the results.
2. Vital sign devices-Using user Meta data and images selecting the best model to analysis the results. We run a model to find type OCR sign and model for OCR on a Seven Segment display.
3. Wound Analysis-Using the calibration sticker, calculate pixel size in MM and color calibration. Run AI model for wound semantic segmentation. Using the blob for calculation of the major axis of the blob, calculating the red mean inside the wound.

### Oral Analysis-Wizard Analysis

Classify the analysis type user needed, give the user extraction how to continue according to the analysis type check the user input quality data.
1. Automated Detection of Environmental: The technology automatically identifies the user's face and mouth area using AI models.
2. Real-time Feedback and Guidance: The technology provides real-time feedback and guidance to the user during the image capture process. Visual indicators, such as on-screen overlays or audio cues, assist users in positioning the camera correctly, adjusting framing, and ensuring optimal shooting conditions for capturing high-quality photos.
3. Adaptive Image Processing: Adaptive image processing algorithms analyze each captured image to identify areas for improvement and automatically apply enhancements to optimize image quality. This includes adjustments to sharpness, noise reduction, color balance, and dynamic range compression, tailored to the specific characteristics of the scen.
   We use AI models for Image quality assessment (IQA).
4. All images taken from the oral cavity that successfully passed the quality tests are automatically sent to the server to run All images taken from the oral cavity that successfully passed the quality tests are automatically sent to the algorithm server to check whether there is a suspicion of a cancerous lesion.

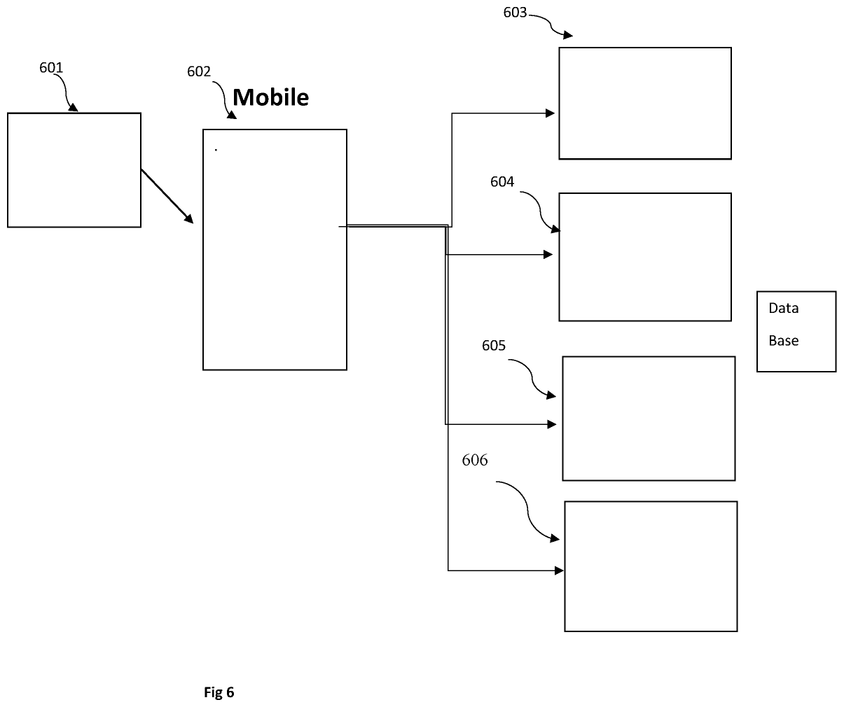

Reference is now made to FIG. 6 illustrating the procedure: 601 Input of Image, user Meta data into the Mobile wizard. The wizard 602 Wizard Analysis

Classify the analysis type user needed, give the user extraction how to continue according to the analysis type. Analysis types in this case are:
603 Vital Sign Device-Device detection classification OCR models and data match.
604 Stoma Analysis-AI Model for semantic segmentation and data extraction classic Computer Vision algorithms
605 Wound Analysis-AI Model for semantic segmentation and data extraction classic Computer Vision algorithms
606 Oral Analysis—

AI model classification and detection cancer.

### Validation of the System and Method of the Present Invention

The following provides a description of the overall validation process that was carried out of the present invention:

Data was extracted from non-transmitting home medical devices, by the methods of the invention herein described, rendering the data accessible. Following a successful pilot with approximately 200 users, the main insights and validation process are as follows:

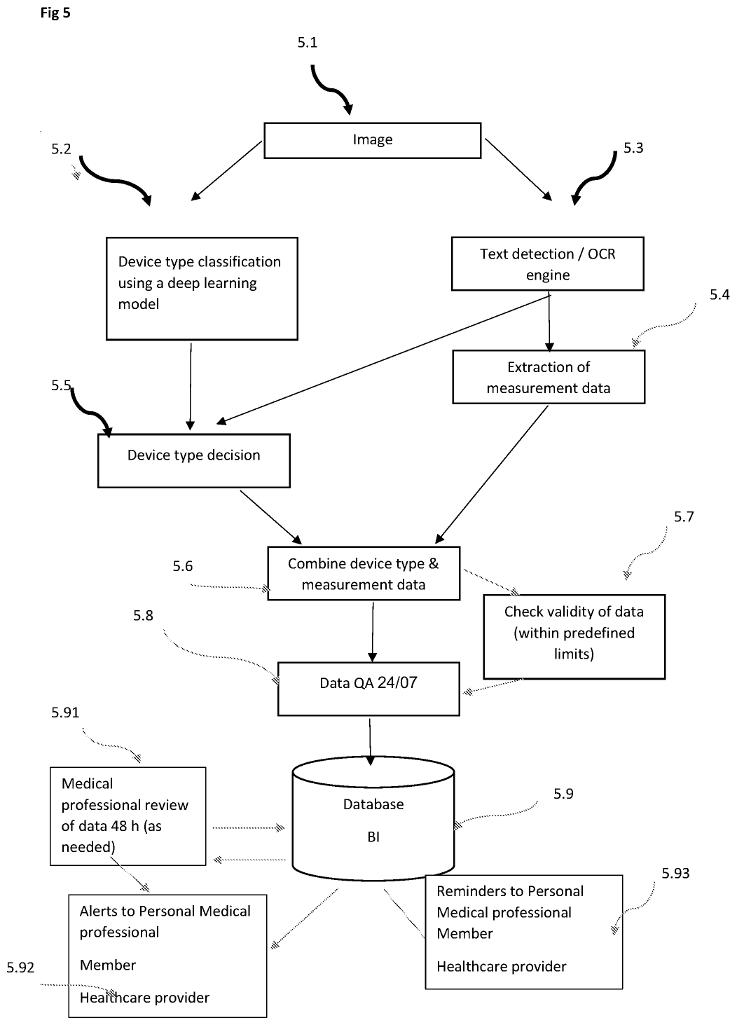

The CatAI system is based on the transmission of the data via the healthcare provider's application, in any of the methods listed below (see FIG. 5):

Photographing the medical device by the patient/family member/caregiver; the application connects to the mobile device camera to take a picture and send the image 5.1 to the system for analysis and data extraction 5.4, Device type classification using a deep learning model 5.2, text detection using an OCR engine 5.3. When the data has been extracted and the device type decision 5,5 has been made, the device type and the measurement data are combined 5.6. Validity of the data is determined by the system according to predefined limits 5.7. The data undergoes quality assurance 5.8 and is then transferred to the healthcare provider's database 5.9. Medical professional review 5.91 of the data was carried out within 48 hours of receipt, and alerts and reminders were sent to the medical professionals, health care providers and members (users) 5.92, 5.93.
Further details of the validation of the present invention are reported below:
The data is automatically entered into the medical file, although in some embodiments of the invention some of the data may be entered manually.
Filling out a questionnaire. In one study, the questionnaire was adapted for the use of congestive heart failure patients. Additional questionnaires have been prepared in accordance with client requests—for diabetics, the elderly without background diseases, orthopedic rehabilitation patients.

The control principles of the transmitted responses:
1. All the transmitted images are checked by the QA personnel within 2 minutes from the time of receipt at the digital control center.
2. The system includes threshold alerts for each measured parameter, based on a standard medical protocol. The alerts are automatically transmitted in real time to the digital control center.
3. A medical professional at the digital control center checks the information every 48 hours, and if necessary, refers also to the real time alerts.
4. Two types of information are checked by the medical professional, firstly whether any information has been sent, and the data itself.
5. “Serious” alerts (for example, hypoglycemia) are dealt with immediately as soon as the information is understood.
6. More than 10,000 images have already been transmitted through the system, raising the device's classification level to over 95%.
7. The data extraction accuracy level of the saturation, diabetes and blood pressure devices is over 90%, when the image is clear and was taken at an appropriate angle.
8. If the device is unidentifiable, the user receives the following response: “Please remeasure” (these images are also checked).
9. Even before the image is received at the digital control center, initial filtering and quality control of the image is performed (in the application), through the identification of numbers or letters. This initial filtering of the image quality ensures that it is “legitimate” and is at a quality level that is decipherable (a mechanism similar to the one used in the banking industry, for example, which ensures the “legitimacy” of the image of a deposited check). This mechanism supplements the values check that is performed based on the parameter and type of device.

The preliminary insights gained from the pilot participants:
1. The patients are acquainted with and accustomed to using their home medical devices—there was no data transmission with measurement errors (error notification appearing on the device).
2. The patients made sure to transmit at least once. Half of them transmitted at least twice.
3. Most of the patients chose photography as their preferred method for transferring data from their home medical devices (taking photos of the measurements as they appear on the devices, especially in the case of the blood pressure, glucose and saturation measurement devices).
4. Most of the pilot participants were over 70 years old. In some cases, a caregiver or family member helped them use the app.
5. We received excellent feedback from the physicians. They mainly emphasized the improved ability to manage and control the patients' medical condition thanks to the app. Some of the physicians stated that the system directed them how to treat the patients more effectively and wisely.
6. The system renders the information accessible digitally-alerts (to the caregiver only/healthcare provider) when no measurement data has been transmitted for more than 24 hours. This allows the caregiver to initiate contact with the patient or their caregiver/family member and inquire how they are feeling, and recommend that they continue transmitting the measurements of their medical parameters.
7. The system enables the healthcare provider to generate alerts (for the caregiver) on deviations from the normal values, for example, alerts regarding a low saturation level, hypoglycemia, etc.).
8. The caregivers stated that thanks to the system, a personal connection was created, and feedback was provided to the patient at their home.
9. The patients also provided positive feedback regarding the connection.
10. The control system displays a different indication of measurements that are entered manually, as opposed to those extracted from the transmitted photo. Our experience shows that when entering data manually, patients tend to enter normal values (such as normal glucose levels, or a weight that is slightly lower than the actual weight).
11. In manual data entry, a significant level of typing errors was detected.
12. Also in the manual data entry, errors were detected in entering the values into the wrong field (for example, saturation level entered into the heart rate field).
13. All the above lead to the conclusion that photographing the measurements is preferable to manual data entry.

### Results:

From a clinical perspective, several instances occurred in which deterioration of patients was detected thanks to the accessibility of the information to the healthcare provider (feedback from the medical provider):
Fluid retention resulting in weight gain.
Desaturation—the patient received an oxygen generator for his use at home.
Anomalous glucose levels that the caregiver was unaware of (in the case of hypoglycemia, the patient was given honey; in the case of hyperglycemia, he received instructions how to control his glucose level).

Additional insight gained from the fact that the group of patients who had participated in the pilot did not use transmitting devices (Bluetooth) indicates that although embodiments of the present invention supports transmitting devices, at this stage it is preferable to receive the medical device measurement data through the transmission of images taken of the older generation devices that are commonly used.

Exemplary qualitative reports from the validation study:

### Senior Caregiver, Sabar Health (Israel)—Home Hospital:

“Thanks to the CatAI system (the system of the present invention), the patients I monitor can maintain constant contact with me. The system allows me to continuously track the medical condition of my patients, and provides me with precise, high-quality information about their medical status in their natural environment.

Using CatAI system (the system of the present invention), I am able to form a reliable and full picture of the medical situation of all my patients. The system directs my attention to those whose condition is deteriorating. The interface is intuitive and easy to use; with one glance, I can see which of my patients needs close monitoring and special attention. For example, when the system alerts to reduced saturation coupled with weight gain, I know that I need to consider changing the patient's medical treatment. Changes in blood pressure and heart rate also indicate problems that need to be addressed without delay.

As a caregiver attending to congestive heart failure patients, early identification of changes in their medical condition enables me to act quickly, and often prevent further deterioration. The system's alerts to changes in vital parameters allow me to make data-driven decisions and immediately adjust the treatment.

Importantly, I find that the patients' response to using the CatAI app is amazing, far beyond my expectations. The process itself is simple and easy for them to execute, the app is extremely user friendly, and they are very pleased to receive my feedback.”

### Patient Testimonial

“Thank you, CatAI. Your app helped/saved my husband.

Years of unstable blood glucose levels have now ended, thanks to you.

Following his stroke, my husband's physicians warned him that he simply must stabilize his blood glucose levels. Upon his discharge from the hospital to home rehab, he started using the CatAI app. His glucose levels were unbalanced, despite having taken prescribed medication for years. With the constant test result reporting via the app, his physician was able to examine his glucose levels at different times of the day, and recommended that he consult with specialists at a diabetes center. These specialists changed my husband's medication regimen based on the glucose test results recorded at various hours of the day, and within several days, his glucose level stabilized at 100.

The ability to continuously record the test results, and his physician's easy access to the data, and importantly—the ability to continue tracking the glucose level after the change in medication, helped us resolve a problem that my husband had been suffering from for years.

Your app is very easy to use, and definitely saves lives.

### Validation Study Conclusions:

The system and method disclosed herein enables and facilitates conducting a reliable digitization process of data in order to reflect the patient's medical condition and support clinical decision making.

Although only a few exemplary embodiments have been described in detail herein, those skilled in the art will readily appreciate that many modifications are possible in the exemplary embodiments without materially departing from the novel teachings and advantages of the embodiments of the present disclosure. Accordingly, all such modifications are intended to be included within the scope of the embodiments of the present disclosure as defined in the following claims. In the claims, means-plus-function clauses are intended to cover the structures described herein as performing the recited function and not only structural equivalents, but also equivalent structures.

The above-disclosed subject matter is to be considered illustrative, and not restrictive, and the appended claims are intended to cover any and all such modifications, enhancements, and other embodiments that fall within the scope of the present invention. Thus, to the maximum extent allowed by law, the scope of the present invention is to be determined by the broadest permissible interpretation of the following claims and their equivalents, and shall not be restricted or limited by the foregoing detailed description.

We claim:

1. A system for acquiring medical data from connected and non-connected devices, and optimizing said data, comprising:
a mobile device containing at least one processor, a memory unit and at least one camera, a server containing at least one processor and a memory unit, wherein:
said mobile device takes an image of a non-connected health monitoring device, transferring the data to a server, said server using computational means to detect the type and data from said device using machine learning modules, categorizing said results and saving the correct results into a database wherein said system is configured to receive images from a dedicated application connected to a specific user mobile device.

2. The system described in claim 1 wherein said system is configured to receive images from a messaging service, connected to a specific user account.

3. The system described in claim 1 wherein the system is configured to send reminders, suggestions and predictions using a chatbot or other to messaging service connected to a specific user.

4. The system described in claim 1 wherein said system is configured to adjust the personal gathered data, adjust suggestions and recommendations and predictions, based on user prior health conditions.

5. The system described in claim 1 wherein said system is configured to collect user health data, apply said data against health protocols, and inform the user regarding the health status based on the results given by said health protocols.

6. The system of claim 1 wherein said system comprises a graphical user interface (GUI) wizard on a mobile phone said wizard comprising instructions for taking pictures of different areas of the body using an AI model to recognize said body areas, imaging vital sign devices, and checking lighting and sharpness of said images, real time identification of lesions by size and color, qualifying images and sending said qualified images to the algorithm server for evaluating degree of risk that said lesion is cancerous.

7. A method for acquiring medical data from connected and non-connected devices, and optimizing said data, comprising steps of
obtaining the system of claim 1, said system
receiving an image from a user, concurrently running identification of the image against a machine learning module configured for detecting a specific type of a non-connected health monitoring device and detecting and extracting text from said image said system further deciding on the correct monitoring device and text and saving said data in a database.

8. The method of claim 7 further comprising steps of parsing captured images of said connected and non-connected devices comprising steps of concurrently subjecting the device type to a deep learning model and thereby classifying said device
detecting text displayed on said device by OCR engine
combining said device classification with detected text
providing a device type decision
extracting measurement data from said text detection
combining device type and measurement data
organizing data into a database

9. The method of claim 7 comprising additional steps of
detecting types of non-connected health monitors by receiving an image data set of different non-connected health monitors
feeding said image data to a deep learning module configured to differentiate between types of heath monitors
creating a validation set of images against said deep learning model and creating an AI model, said AI model providing a quality score.

10. The method described in claim 7 comprising further steps of receiving images from a dedicated application connected to a specific user mobile device.

11. The method described in claim 7 comprising further steps of can be receiving images from a messaging service, connected to a specific user account.

12. The method described in claim 7 comprising steps of sending reminders, suggestions and predictions using a chatbot or other to messaging service connected to a specific user.

13. The method described in claim 7 comprising steps of adjusting the personal gathered data, and adjusting suggestions and predictions, based on user prior health conditions.

14. The method described in claim 7 comprising steps of collecting user health data, applying said data against health protocols, and informing the user, as well as others, regarding the health status based on the results given by said health protocols.

15. A method for optimizing a user collected health data wherein:
data is collected by the system of claim 1 concerning a user from both connected and non-connected devices, optimal data is continuously computed, correcting data received from connected devices to get accurate health data for a specific user.

16. The method described in claim 15 wherein said data is further optimized by receiving specific health conditions associated with a specific user.

17. The method described in claim 15 wherein said received data from connected devices is normalized based on prior data collected about a user from both connected and non-connected devices.

18. The method described in claim 15 wherein said data is used to provide alerts, suggestions, and predictions regarding the health status of an individual.

19. The method of claim 15 further comprising steps of providing a graphical user interface (GUI) wizard on a mobile phone said wizard comprising instructions for taking pictures of different areas of the body using an AI model to recognize said body areas, imaging vital sign devices, and checking lighting and sharpness of said images, real time identification of lesions by size and color, qualifying images and sending said qualified images to the algorithm server for evaluating degree of risk that said lesion is cancerous.
